(19) United States
(12) Patent Application Publication
(10) Pub. No.: US 2019/0133941 A1
(43) Pub. Date: May 9, 2019

(54) STABLE PROBIOTIC SUSPENSIONS

(71) Applicant: DSM IP Assets B.V., TE Heerlen (NL)

(72) Inventors: Seema K. MODY, Durham, CT (US); Syed H. NAQVI, Stamford, CT (US)

(21) Appl. No.: 16/304,500

(22) PCT Filed: Jun. 21, 2017
(86) PCT No.: PCT/US2017/038452

§ 371 Date: Nov. 26, 2018

Related U.S. Application Data
(60) Provisional application No. 62/353,376, filed on Jun. 22, 2016.

(51) Int. Cl. A61K 9/10; A61K 9/14; A61K 36/899; A61K 36/28; A61K 36/54; A61K 35/644; A61K 36/889; A61K 35/747; A61P 1/14

(52) U.S. Cl. CPC A61K 9/10; A61K 9/14; A61K 36/899; A61K 36/28; A61P 1/14; A61K 35/644; A61K 36/889; A61K 35/747; A61K 36/54

(57) ABSTRACT

Stable probiotic-containing suspensions are disclosed that comprise at least one edible oil, at least one edible wax or edible shear thickening agent, and at least one probiotic powder. The improved suspensions have significantly reduced visual settling of the probiotic powder while maintaining acceptable water activity levels and a user-friendly viscosity.

### CROSS REFERENCE TO RELATED APPLICATIONS

This application claims the benefit of the filing date of U.S. Provisional Patent Application No. 62/353,376 filed Jun. 22, 2016, the disclosure of which is hereby incorporated by reference in its entirety.

### BACKGROUND OF THE INVENTION

Probiotics are live microbial organisms in foods and supplements which beneficially affect the host by supporting naturally occurring gut flora, by competing with harmful microorganisms in the gastrointestinal tract, by assisting useful metabolic processes, and by strengthening the resistance of the host organism against toxic substances. A number of probiotic organisms are used in foods, examples being the bacterial genera Lactobacillus or Bifidobacterium, or bacterial species Bifidobacterium lactis, Lactobacillus rhamnosus or strains Lactobacillus paracasei St 11 (or NCC2461), Lactobacillus johnsonii Lai (=Lactobacillus LCI, Lactobacillus johnsonii NCC533). Probiotic organisms should survive for the lifetime of the product in order to be effective, and furthermore they should survive the entire gastrointestinal tract to the colon. Probiotic organisms can be incorporated into dairy products such as yogurts. The need is felt to deliver the beneficial microorganisms in other foodstuff types, for example in liquid-based products especially those which undergo heat treatment in at least one stage of their preparation. The main problem in preparing liquid-based health foods is the combination of high temperature and water that may destroy all, or a significant portion, of the included probiotics.

US 2016/0081940 A1 describes a method of preparing microorganisms including probiotics by encasing them in a starch based polymer shell, thereby extending the shelf life of the microorganism. The problem with this approach, however, is that an additional heating step is required during formula preparation to dissolve the starch shell and activate the microorganism activity. The likelihood is high of too little or too much thermal energy being introduced. If too little thermal energy is introduced, the starch shell will not dissolve and the microorganism will not be activated. If too much energy is introduced, the microorganism will likely be destroyed.

US2005/0019417 A1 describes a method of preparing products containing moisture-sensitive living microorganisms including probiotics, comprised of steps through which a suspension of probiotics and a sugar polymer in a water miscible solvent is sprayed onto water soluble, gel-forming solid particles. By these means, the core, composed of water soluble gel-forming solid particles, may absorb solvent residues and provide protection to probiotics placed onto the solid core. This approach involves a rather convoluted multi-step process to solve the problem and it is expected that the presence of such hygroscopic materials could actually prematurely activate the life cycle of the microorganism if environmental moisture is introduced during storage, thereby significantly shortening the shelf life of the product.

Probiotic potency is typically expressed as “colony forming units”, commonly known as cfus. It is common to freeze dry or use other modes to almost eliminate the water from the live probiotic cultures to make them dormant and shelf stable for very long periods of time. Similarly, some probiotic bacteria and yeasts may be in a dormant spore form.

Oil suspensions of probiotics are also commercially known. However, such suspensions are very limited in the number of probiotic units that can be incorporated since, at higher level, the probiotic powder will agglomerate and clump so as to render the product inhomogeneous and not useful.

Both of the publications described also involve extra steps on the part of the rather distracted parental user and is also thereby perceived as undesirable. Further, coating the probiotics creates an incorporation step that is very sensitive to variability in heat and the uneducated user will likely improperly incorporate and activate the probiotic in the face of multiple distractions; resulting in significant variability in the measured dose due to poor distribution of the probiotic powders in the vehicle.

What is needed, but presumably lacking, is a mode of stabilizing the probiotic powders while maintaining a long active shelf life of the probiotic suspension while providing an easy method of dispensing and incorporating the probiotic product without difficulty or extra steps on the part of the user.

### SUMMARY OF THE INVENTION

A stable suspension of probiotic powder in an oil and wax combination that is easily digestible for an infant is provided. The powder is stably suspended such that shaking the suspension by the parent is unnecessary since the powder does not settle to the bottom as ordinary products are known to do. Hence, there is no significant variability in the measured dose due to poor distribution of the probiotic powders in the vehicle. Further, the viscosity of the probiotic powder and oil and wax suspension vehicle is designed such that dose measuring and incorporation is reliable and repeatable as well as quickly and easily done.

The present invention is directed to a stable probiotic-containing suspension comprising:
i) at least one edible oil;
ii) at least one edible wax; and
iii) a probiotic powder;
   wherein the stable probiotic-containing suspension has a visual settling score of 3 or less when stored for more than one week at 0° C. to 40° C.; and wherein the water activity of the stable probiotic-containing suspension is between 0.001 to 0.40 a_{w} when run in the water activity test.

In one embodiment, the at least one edible oil, the at least one edible wax, and the probiotic power in the stable probiotic-containing suspension are all fit for human consumption in their composition and purity levels.

In another embodiment, the stable probiotic-containing suspension has a visual settling score of 3 or less when stored for more than two weeks at 0° C. to 40° C.

In another embodiment, the stable probiotic-containing suspension has a visual settling score of 3 or less when stored for more than five weeks at 0° C. to 40° C.

In one embodiment, the edible oil is selected from the group of rice bran oil, sunflower oil, corn oil, canola oil, avocado oil, olive oil, safflower oil, soybean oil, peanut oil, sesame oil, cottonseed oil, coconut oil, grape seed oil, palm oil, flaxseed oil, rapeseed oil, or combinations thereof.

In one embodiment, the edible wax is selected from the group of carnauba wax, beeswax, candelilla wax, ceresine wax, microcrystalline wax, ozokerite wax, paraffin wax, cetyl palmitate, or combinations thereof.

In one embodiment, the probiotic powder is selected from the group of Lactobacillus, Bifidobacterium, Bacillus, Streptococcus, Saccharomyces, Enterococcus, Staphylococcus, or combinations thereof.

In one embodiment, the stable probiotic-containing suspension has a_{w} that is between 0.001 to 0.35 a_{w}. In another embodiment, the stable probiotic-containing suspension has a_{w} that is between 0.001 to 0.30 a_{w}.

In one embodiment, the viscosity of the stable probiotic-containing suspension is between 100 to 100000 centipoises. In another embodiment, the viscosity of the stable probiotic-containing suspension is between 100 to 6000 centipoises. In another embodiment, the viscosity of the stable probiotic-containing suspension is between 100 to 3000 centipoises.

The present invention is also directed to a stable probiotic-containing suspension comprising:
i) at least one edible oil;
ii) at least one edible shear thickening additive; and
iii) a probiotic powder;
   wherein the stable probiotic-containing suspension has a visual settling score of 3 or less when stored for more than one week at 0° C. to 40° C.; and wherein the water activity of the stable probiotic-containing suspension is between 0.001 to 0.40 a_{w}.

In one embodiment, the at least one edible oil, the at least one edible wax, and the probiotic power in the stable probiotic-containing suspension are all fit for human consumption in their composition and purity levels.

In another embodiment, the stable probiotic-containing suspension has a visual settling score of 3 or less when stored for more than two weeks at 0° C. to 40° C.

In another embodiment, the stable probiotic-containing suspension has a visual settling score of 3 or less when stored for more than five weeks at 0° C. to 40° C.

In one embodiment, the edible oil is selected from the group of rice bran oil, sunflower oil, corn oil, canola oil, avocado oil, olive oil, safflower oil, soybean oil, peanut oil, sesame oil, cottonseed oil, coconut oil, grape seed oil, palm oil, flaxseed oil, rapeseed oil, or combinations thereof.

In one embodiment, the edible shear thickening additive is selected from the group of gums, polysaccharides, collagens, starches, or combinations thereof.

In one embodiment, the probiotic powder is selected from the group of Lactobacillus, Bifidobacterium, Bacillus, Streptococcus, Saccharomyces, Enterococcus, Staphylococcus, or combinations thereof.

In one embodiment, the stable probiotic-containing suspension has a_{w} that is between 0.001 to 0.35 a_{w}. In another embodiment, the stable probiotic-containing suspension has a_{w} that is between 0.001 to 0.30 a_{w}

In one embodiment, the viscosity of the stable probiotic-containing suspension is between 100 to 100000 centipoises. In another embodiment, the viscosity of the stable probiotic-containing suspension is between 100 to 6000 centipoises. In another embodiment, the viscosity of the stable probiotic-containing suspension is between 100 to 3000 centipoises.

The present invention is also directed to a method of making the stable probiotic-containing suspension, comprising the steps of:
a. Adding an edible oil to a mixing vessel;
b. Heating the vessel to above the melting point of the edible wax;
c. Adding the edible wax to the mixing vessel;
d. Agitating with an appropriate agitator until the oil and wax mixture is homogenous and shows no visible striations;
e. Cooling the oil and wax mixture to below 30° C.;
f. Adding a probiotic powder; and
g. Agitating the mixture until the probiotic powder is well suspended.

In one embodiment, in step b of the above-mentioned method, the vessel is heated to at least 5° C. above the melting point of the edible wax.

The present invention is also directed to a method of making the stable probiotic-containing suspension, comprising the steps of:
a. Adding a 5-99% of the total amount of edible oil to be used to a mixing vessel;
b. Heating the vessel to above the melting point of an edible wax;
c. Adding the edible wax to the mixing vessel;
d. Agitating with an appropriate agitator until the oil and wax mixture is homogenous and shows no visible striations;
e. Add the remaining 95-1% edible oil to be used to the mixing vessel to decrease the temperature of the oil/wax mixture, continue to mix until mixture becomes homogeneous.
f. Cooling the oil and wax mixture to below 30° C.;
g. Adding a probiotic powder; and
h. Agitating the mixture until the probiotic powder is well suspended.

In one embodiment, in step b of the above-mentioned method, the vessel is heated to at least 5° C. above the melting point of the edible wax.

### BRIEF DESCRIPTION OF THE DRAWINGS/FIGURES

FIG. 1a shows an example of a probiotic-containing suspension with a visual settling score of 0.

FIG. 1b shows an example of a probiotic-containing suspension with a visual settling score of 1.

FIG. 1c shows an example of a probiotic-containing suspension with a visual settling score of 2.

FIG. 1d shows an example of a probiotic-containing suspension with a visual settling score of 3.

FIG. 1e shows an example of a probiotic-containing suspension with a visual settling score of 4.

FIG. 1f shows an example of a probiotic-containing suspension with a visual settling score of 5.

### DETAILED DESCRIPTION OF THE INVENTION

Unless defined otherwise, all technical and scientific terms used herein have the same meaning as commonly understood by one of ordinary skill in the art to which this invention belongs. Generally, the nomenclature used herein and the laboratory procedures are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well-known and commonly employed in the art.

The features and advantages of the invention may be more readily understood by those of ordinary skill in the art upon reading the following detailed description. It is to be appreciated that certain features of the invention that are, for clarity reasons, described above and below in the context of separate embodiments, may also be combined so as to sub-combinations thereof.

Embodiments identified herein as exemplary are intended to be illustrative and not limiting.

In this specification and in the claims that follow, reference will be made to a number of terms, which shall be defined to have the following meanings:

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein.

“Optional” or “optionally” means that the subsequently described event or circumstance can or cannot occur, and that the description includes instances where the event or circumstance occurs and instances where it does not.

An “oil”, as used herein, refers to edible oils.

A “wax”, as used herein, refers to edible waxes.

A “probiotic”, as used herein, is an oral supplement, a food product or a over the counter drug that contains a sufficient number of viable microorganisms to deliver beneficial health effects.

A “probiotic powder”, as used herein, is the actual bacteria and/or yeast in dry form along with any nutrients included for the purpose of sustaining the colonies once they are activated.

“Probiotic concentration” is the amount of colony forming units or cfus of probiotic units per milliliter of suspension.

A “administered dose amount” is the volume or weight of probiotic powder-containing suspension administered to the person.

“Water activity”, as used herein, is the partial vapor pressure of water in the suspension divided by the standard state partial vapor pressure of water. In the field of food science, the standard state partial vapor pressure of water is most often defined as the partial vapor pressure of pure water at the same temperature. The unit for Water Activity is a_{w} or equilibrium relative humidity (ERH). Low water activities are preferred for probiotics since the presence of higher amounts of water activate the probiotic life cycle and shorten its effective lifetime during storage. A thorough discussion of water activity and its measurement can be found at: <http://www.fda.gov/ICECl/Inspections/InspectionGuides/InspectionTechnicalGuides/ucm0729 16.htm>

A “stable probiotic-containing suspension”, as used herein, refers to a solution of oil and wax and probiotic powder which, once created, will not be subject to significant separation or significant settling of ingredients nor will it significantly activate the life cycle of the bacteria or yeast.

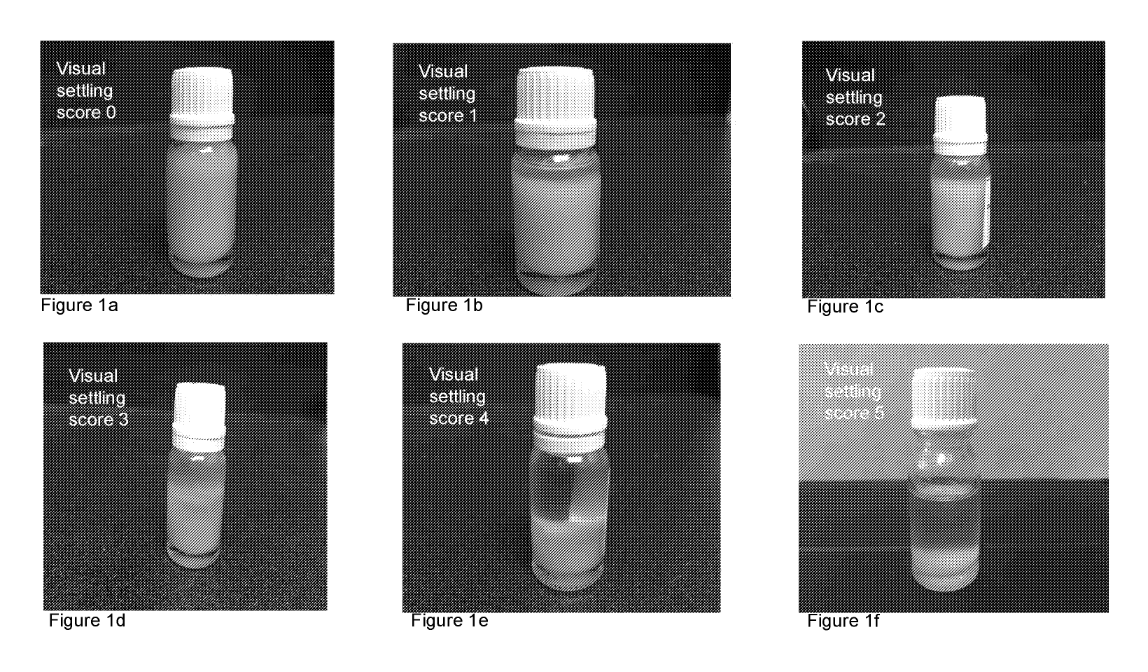

A “visual settling score” is a value assigned to probiotic powder containing liquids after it has been stored at ambient temperatures for one to eight weeks or more. The following scale is used to assess the degree of settling at the bottom of the glass jars in each sample at each time period and assigned a visual settling score:
0=No visual settling of probiotic particles in the probiotic-containing liquid suspension (FIG. 1a);
1=Partial settling of probiotic particles in the probiotic-containing liquid suspension, where the fraction of settling probiotic particles account for no less than 13/14 volume of the total suspension (FIG. 1b);
2=Partial settling of probiotic particles in the probiotic-containing liquid suspension, where the fraction of settling probiotic particles account for less than 13/14 but more than 11/14 volume of the total suspension (FIG. 1c);
3=Partial settling of probiotic particles in the probiotic-containing liquid suspension, where the fraction of settling probiotic particles account for less than 11/14 but more than 8/14 volume of the total suspension (FIG. 1d);
4=Partial settling of probiotic particles in the probiotic-containing liquid suspension, where the fraction of settling probiotic particles account for less than 8/14 but more than 5/14 volume of the total suspension (FIG. 1e);
5=Partial settling of probiotic particles in the probiotic-containing liquid suspension, where the fraction of settling probiotic particles account for less than 5/14 but no more than 3/14 volume of the total suspension (FIG. 1f).

In one embodiment, a stable probiotic-containing suspension comprises:
i) at least one consumable oil;
ii) at least one consumable wax; and
iii) a probiotic powder;
   wherein the stable probiotic-containing suspension has a visual settling score of 3 or less after storage for more than one week at 0° C. to 40° C.; more preferably, 0° C. to 30° C.; most preferably, 0° C. to 25° C.; wherein the water activity of the stable probiotic-containing suspension is between 0.001 to 0.40 a_{w} when run in the water activity test; between 0.001 to 0.35 a_{w}; between 0.001 to 0.30 a_{w}; between 0.001 to 0.25 a_{w}; between 0.001 to 0.20 a_{w}.

In another embodiment, the viscosity of the stable probiotic-containing suspension is in the range of 100 to 100,000 centipoises; more preferably in the range of 100 to 6000 centipoises; more preferably in the range of 100 to 3000 centipoises; and most preferably 100 to 800 centipoises.

In one embodiment, the at least one edible oil, the at least one edible wax, and the probiotic power in the stable probiotic-containing suspension are all fit for human consumption in their composition and purity levels.

In another embodiment, the stable probiotic-containing suspension has a visual settling score of 3 or less when stored for more than two weeks at 0° C. to 40° C.

In another embodiment, the stable probiotic-containing suspension has a visual settling score of 3 or less when stored for more than five weeks at 0° C. to 40° C.

Non-limiting examples of oils include rice bran oil, sunflower oil, corn oil, canola oil, avocado oil, olive oil, safflower oil, soybean oil, peanut oil, sesame oil, cottonseed oil, coconut oil, grape seed oil, palm oil, flaxseed oil, rapeseed oil, other so-called vegetable oils, oils enriched with omega 3 fatty acids such as fish oils, or oil from microalgae, or other useful oils that are approved by the FDA in the Code of Federal Regulations Title 21, Part 172 and combinations thereof.

Non-limiting examples of waxes fit for human consumption include rice bran wax, petroleum waxes, carnauba wax, beeswax, candelilla wax, ceresine wax, microcrystalline wax, ozokerite wax, paraffin wax, cetyl palmitate, or other useful waxes that are approved by the FDA in the Code of Federal Regulations Title 21, Part 172, and combinations thereof.

Non-limiting examples of probiotics are of the genus Lactobacillus, Bifidobacterium, Bacillus, Enterococcus, Staphylococcus, and Streptococcus. Specific non-limiting examples of strains of these genera are Lactobacillus rhamnosus GG (LGG), Bifidobacterium lactis BB-12, and Streptococcus thermophilus ST-21, and combinations thereof. Some yeasts are also useful as probiotics and are sometimes included in the probiotic formulations. One non-limiting example of a yeast used in probiotics is Saccharomyces boulardii.

With the teachings herein, it is to be understood that a person of ordinary skill in the art can select from these lists freely to provide desired combinatorial effects such as viscosity, settling, color and the like and, as such, it is contemplated that multiple combinations not expressly defined herein could be useful and are considered as part of this teaching.

In another embodiment, a stable probiotic-containing suspension comprises:
i) at least one edible oil;
ii) at least one edible shear thickening additive;
iii) a probiotic powder;
   wherein the probiotic-containing suspension has a visual settling score of 3 or less after storage for more than a week at 0° C. to 40° C.; more preferably, 0° C. to 30° C.; most preferably, 0° C. to 25° C.; wherein the water activity of the stable probiotic-containing suspension is between 0.001 to 0.40 a_{w} when run in the water activity test; between 0.01 to 0.35 a_{w}; between 0.01 to 0.30 a_{w}; between 0.01 to 0.25 a_{w}; between 0.01 to 0.20 a_{w}.

In one embodiment, the viscosity of the stable probiotic-containing suspension is in the range of 100 to 10000 centipoises; more preferably in the range of 100 to 6000 centipoises; most preferably in the range of 100 to 3000 centipoises. In one embodiment, the at least one edible oil, the at least one edible wax, and the probiotic power in the stable probiotic-containing suspension are all fit for human consumption in their composition and purity levels.

In another embodiment, the stable probiotic-containing suspension has a visual settling score of 3 or less when stored for more than two weeks at 0° C. to 40° C.

In another embodiment, the stable probiotic-containing suspension has a visual settling score of 3 or less when stored for more than five weeks at 0° C. to 40° C.

Non-limiting examples of oils include rice bran oil, sunflower oil, corn oil, canola oil, avocado oil, olive oil, safflower oil, soybean oil, peanut oil, sesame oil, cottonseed oil, coconut oil, grape seed oil, palm oil, flaxseed oil, rapeseed oil, other so-called vegetable oils, oils enriched with omega 3 fatty acids such as fish oils, oil from microalgae, or other useful oils that are approved by the FDA in the Code of Federal Regulations Title 21, Part 172, and combinations thereof.

Non-limiting examples of shear thickening additives fit for human consumption are gums, polysaccharides, collagens, starches, silicas or other useful shear thickening additives, and combinations thereof that are at least partially soluble in the hydrophobic environment provided by the oils and are approved by the FDA in the Code of Federal Regulations Title 21, Part 172. It is contemplated that, with the teaching provided herein, a person having ordinary skill in the art would be able to competently choose oil and shear thickening additive combinations that would be effective and have other desirable properties.

Non-limiting examples of probiotics are of the genus Lactobacillus, Bifidobacterium, Bacillus, Enterococcus, Staphylococcus, and Streptococcus. Specific non-limiting examples of strains of these genera are Lactobacillus rhamnosus GG (LGG), Bifidobacterium lactis BB-12, and Streptococcus thermophilus ST-21, and combinations thereof. Some yeasts are also useful as probiotics and are sometimes included in the probiotic formulations. One non-limiting example of a yeast used in probiotics is Saccharomyces boulardii.

Probiotic concentration in the contemplated suspension can be from 10 million cfu/mL to 100 billion cfu/mL, from 10 million to 50 million cfu/mL, more preferably from 50 million to 100 million cfu/mL, from 100 million to 500 million cfu/mL, from 500 million to 1 billion cfu/mL, from 1 billion to 5 billion cfu/mL, from 5 billion to 10 billion cfu/mL, from 10 billion to 15 billion cfu/mL, from 15 billion to 20 billion cfu/mL, from 20 billion to 25 billion cfu/mL, from 25 billion to 30 billion cfu/mL, from 30 billion to 35 billion cfu/mL, from 35 billion to 40 billion cfu/mL, from 40 billion to 45 billion cfu/mL, from 45 billion to 50 billion cfu/mL, from 50 billion to 55 billion cfu/mL, from 55 billion to 60 billion cfu/mL, from 60 billion to 65 billion cfu/mL, from 65 billion to 70 billion cfu/mL, from 70 billion to 75 billion cfu/mL, from 75 billion to 80 billion cfu/mL, from 80 billion to 85 billion cfu/mL, from 85 billion to 90 billion cfu/mL, from 90 billion to 95 billion cfu/mL, from 95 billion to 100 billion cfu/mL.

The administered dose amount can also vary from 0.2 mL to 5 mL, from 0.2 mL to 0.5 mL, from 0.5 mL to 1.0 mL, from 1.0 mL to 1.5 mL, from 1.5 mL to 2.0 mL, from 2.0 mL to 2.5 mL, from 2.5 mL to 3.0 mL, from 3.0 mL to 3.5 mL, from 3.5 mL to 4.0 mL, from 4.0 mL to 4.5 mL, from 4.5 to 5.0 mL.

With the teachings herein, it is to be understood that a person of ordinary skill in the art can select from these lists freely to provide desired combinatorial effects such as viscosity, settling, color and the like and, as such, it is contemplated that multiple combinations not expressly defined herein but could be made routinely based on the disclosure above are considered as part of this teaching.

In another embodiment, the amount of edible oil in a contemplated suspension can be from 85% to 95%; more preferably from 85% to 86%, 86% to 87%, 87% to 88%, 88% to 89%, 89% to 90%, 90% to 91%, 91% to 92%, 92% to 93%, 93% to 94%, 94% to 95%, 95% to 96%, 96% to 97%, 97% to 98%, 98% to 99% by weight (w/w). The amount of edible wax or edible shear thickening additive in a contemplated suspension can be from 0.5% to 10.0%; more preferably from 0.5% to 1.0%, 1.0% to 1.5%, 1.5% to 2.0%, 2.0% to 2.5%, 2.5% to 3.0%, 3.0% to 3.5%, 3.5% to 4.0%, 4.0% to 4.5%, 4.5% to 5.0%, 5.0% to 5.5%, 5.5% to 6.0%, 6.0% to 6.5%, 6.5% to 7.0%, 7.0% to 7.5%, 7.5% to 8.0%, 8.0% to 8.5%, 8.5% to 9.0%, 9.0% to 9.5%, 9.5% to 10.0% by weight (w/w). The amount of probiotic powder in a contemplated suspension can be from 0.5% to 10.0%, more preferably from 0.5% to 1.0% 1.0% to 2.0%, 2.0% to 3.0%, 3.0% to 4.0%, 4.0% to 5.0%, 5.0% to 6.0%, 6.0% to 7.0%, 7.0% to 8.0%, 8.0% to 9.0%, 9.0% to 10.0% by weight (w/w).

In another embodiment, the visual settling score of the stable probiotic-containing suspension is 3 or less, 2 or less, 1 or less, or 0 after one week, after two weeks, after three weeks, after four weeks, after two months, after three months, after four months, after five months, after six months, or after one year of storage.

In another embodiment, the stable probiotic-containing suspension shelf stability is improved as observed by the visual settling scores after one week, two weeks, three weeks, one month, two months, four months, six months, one year, or two years of storage.

In another embodiment, the blending process of the ingredients is contemplated. Since the wax or shear thickening agent can be difficult to incorporate, especially due to their special physical state, special steps could be taken to ensure proper improved incorporation of these components. This embodiment can be summarized thusly:

Weigh the oil into a heating vessel having an agitator selected from the group of axial flow or radial flow impellors and any combination of these.
1. Heat the oil to above the wax melting point while agitating
2. When temperature reaches above the wax melting point, add wax.
3. Mix until wax is melted completely and mixture become homogeneous. There should be no visible striations in the liquid. The presence of these striations indicated inadequate mixing.
4. Turn off the heat and stop agitation.
5. Let the mixture cool to below 30° C.
6. When mixture is cooled add the probiotics, vitamins, or any other active raw materials.
7. Agitate using a solid suspension agitator such as an axial flow agitator type.
8. After all solids are homogenously suspended in the wax/oil media, cease agitation and commence with any relevant post processing such as filtration and packaging.

In one embodiment, in steps 1 and 2 of the above-mentioned method, the vessel is heated to at least 5° C. above the melting point of an edible wax.

In one embodiment, the wax can be replaced by a shear thickening agent.

In other embodiments, the method of the present invention makes it possible to disperse powdered probiotics uniformly in oil/wax suspension products, without any need for a special apparatus or a high shearing force. In the manufacturing method of the present invention, for example, oil wax suspension can be manufactured using either a batch system or a continuous system.

In one example of such an embodiment, the blending method can be optimized to reduce batch time. This is contemplated as follows:
1. Weigh 5-99% w/w of the oil required in the batch into a heating vessel to minimum batch size having an agitator selected from the group of axial flow or radial flow impellors and any combination of these.
2. Heat the oil to above the wax melting point while agitating.
3. When temperature reaches above the wax melting point, add wax.
4. Mix until wax is melted completely and mixture become homogeneous. There should be no visible striations in the liquid. The presence of these striations indicated inadequate mixing.
5. Add the remaining 95-1% oil to decrease the temperature of the oil/wax mixture, continue to mix until mixture becomes homogeneous.
6. Turn off the heat and stop agitation.
7. Let the mixture cool to below 30° C.
8. When mixture is cooled add the probiotics, vitamins, or any other active raw materials.
9. Agitate using a solid suspension agitator such as an axial flow agitator type.
10. After all solids are homogenously suspended in the wax/oil media, cease agitation and commence with any relevant post processing such as filtration and packaging.

In one embodiment, in step 2 of the above-mentioned method, the vessel is heated to at least 5° C. above the melting point of an edible wax.

In another embodiment, the heating step in the above-mentioned methods is performed after oil and wax, or oil and shear thickening agent, are added to the container.

In another embodiment, the manufacturing methods are modified to be made in liquid processing equipment that is specifically designed for mixing and blending processes involving powders and liquids. An example is the VacuShear Liquid Processor <http://www.abprocess.com/literature/white-papers/the-vacushear-liquid-processor-an-effective-and-ergonomic-approach-to-mixing/<. Adjustments are made to the manufacturing embodiments disclosed herein according to the equipment manufacturer's instructions and the knowhow that a person having ordinary skill would have after receiving the teachings herein.

In another embodiment, the method of manufacturing is adjusted for a continuous process. Adjustments are made to the manufacturing embodiments disclosed herein according to the equipment manufacturer's instructions and the knowhow that a person having ordinary skill would have after receiving the teaching herein.

The stable suspension of probiotic powder described in this application makes it possible to reliably and accurately dispense a dose of probiotics to consumer at the time of use, because it avoids resuspension of probiotic power after settling in the container.

In one embodiment, the consumer is dosed directly from the syringe or dropper provided with the packaging.

In another embodiment, the probiotic suspension is added to the baby formula mixture, juice, breast milk, water, and any other liquids as infant/baby can consume as it is being prepared.

In another embodiment, the probiotic suspension is added to baby food, for consumption by an infant/baby or any other common type of food or drink for consumption by an adult.

It is contemplated that, with the teachings herein, a person skilled in the art may add other known consumable ingredients, such as vitamin supplements, nutritive oils, nutritive fatty acids or fatty esters, and the like, to achieve known desirable effects. These embodiments are also intended for consideration without limitation.

Although various embodiments of the invention have been described using specific terms, devices, and methods, such description is for illustrative purposes only. The words used are words of description rather than of limitation. It is to be understood that changes and variations may be made by those skilled in the art without departing from the spirit or scope of the present invention, which is set forth in the following claims. In addition, it should be understood that aspects of the various embodiments and preferred ranges may be interchanged either in whole or in part and/or be combined in any manners. Therefore, the spirit and scope of the appended claims should not be limited to the description of the preferred versions contained therein.

### Examples

### Test Methods

### Viscosity as a Function of Storage Time

Viscosity was measured according to ASTM D 445 using a Brookfield DV-II+ Pro viscometer and RV spindles #3 at 100 rpm at 25.0° C.+/−0.5° C. Results were recorded as centipoise or mPa·sec.

The standard operating procedure for operating the Brookfield DV-II+ Pro is summarized as follows:

Auto zero—Turn on the viscometer and allow standing on the auto zero setting. After a few seconds the screen appears which indicates 2 digits. Press the key. The screen displays the instruction to remove spindle. After removing the spindle and pressing the key the instrument begins. The viscometer is now auto zeroed.

Test sample preparation—After approximately 15 second the screen displays the instruction to replace spindle. Attach the spindle to viscometer by screwing them on the lower shaft using left hand thread. Press select spindle key and up and down arrow keys. When the desired code is displayed release the arrow key. For all the examples in this application, the #3 spindle was used. To select the spindle for use on the viscometer, first press either up and down key which cause the area to show current speed, press the set speed key for adjusting the speed. Insert the center of this spindle in the test material until the fluid level is at the immersion groove on the spindle shaft. Tilt the spindle slightly while immersing to avoid air entrapment. If the chosen spindle/speed results in torque above 100% meaning high viscosity, reduce the speed value or select a smaller spindle size. Allow time for the individual reading to stabilize, and then record the value. Press the motor on/off/escape key to turn off motor.

### Visual Settling Score of Probiotic Particles in Liquid Suspension as a Function of Storage Time

Visual settling was run using a visual test and score. Samples of the mixed probiotic particle suspension were poured into wide-mouthed 4-ounce glass jars. The suspension samples were all somewhat opaque. The samples were allowed to stand in an upright position for a set period of time at 25° C.+/−5° C. and 60% RH+/−10% RH. The samples were visually examined after two weeks and after 5 weeks. The following scale was used to assess the degree of settling at the bottom of the glass jars in each sample at each time period and assign a visual settling score as defined earlier in this application.

Note that there are always small amounts of observed individual particles hovering near the bottom.

### Visual Separation of Oil and Wax

One less desirable outcome of the above test is the case where the wax and oil themselves start separating from each other over time. The particles may still be suspended in one phase, however, the separation of the oil and wax from each other is also less desirable. This can be readily seen during the visual settling of probiotic particles in liquid suspension test as a typical phase separation between the wax and the oil. While it is undesirable, it is typically overcome in the field by simply shaking the mixture before use. It is a preferred embodiment not to have such a situation, nevertheless, it is still contemplated as an embodiment. If such separation occurs, it is noted in Table 8 as a “Yes” whereas, if it does not occur, it is noted as a “No”.

### Water Activity in Foods

All samples were measured on an AquaLab Series 3® Water Activity Meter according to the AquaLab Series 3® Water Activity Meter Standard Operating Procedure version.

The instrument was setup according to the instructions as described below, and the performance verification, verification, linear offset procedure, and sampling procedure were all run as detailed below.

The sampling procedure is summarized as follows:

Make sure that the sample to be measured is representative of the product. Place the sample in a disposable sample cup, completely covering the bottom of the cup, if possible. Do not fill the sample cup more than half full. Make sure that the rim of the sample cup is clean. Turn the knob to the OPEN/LOAD position and slide the drawer open. Place the prepared sample cup in the drawer and carefully slide the drawer closed. Turn the knob to the READ position. This will seal the chamber and start the reading. In 1 to 2 minutes, the first a_{w} reading will be displayed on the LCD. Length of read times may vary depending on temperature differences between the sample chamber and the sample, and other properties of the sample. When the AquaLab is finished measuring the sample, it will beep (if audible notification is enabled) and the a_{w} and the sample temperature will be displayed on screen. Remove sample when finished sampling. The critical factors in the control of water activity as an adjuvant in preservation are the ingredients in the final product and their effect on water binding capacity which is measured by the ERH (water activity, a_{w}).

### Example 1—Preparation of Wax and Oil Suspension Base for Samples SM 1-SM 12

Since probiotic powders are living organisms, they are readily destroyed by the heat required to melt the wax in the embodiments that are contemplated. As explained in the manufacturing embodiments, therefore, it is contemplated that the steps of mixing the edible melted oil and edible melted wax components are performed separately from the probiotic powder and then subsequently cooled to avoid destroying the probiotic powders. Example 1 focuses on the creation of these stock suspension media (SM). For all stock suspension media sample SM 1-SM-12, total batch size for each example was 1000.00 grams of sample suspension. The selected edible oil was weighed into a beaker and a magnetic string bar was placed into the beaker. The correct ratios of oil and wax can be found in Tables 1 and 2. The beaker was placed on a IKA RET® Basic heating plate with automatic temperature field back control (IKA ETS-D5) which also had a string bar agitator capability. The temperature was set at 5° C. to 15° C. above the melting point of the wax and the feedback cut off setting was set at 5° C. below that set point to have the temperature not go above 15° C. above the wax melting point. Set the stirring agitator bar setting so that it creates a small vortex. The vortex will increase in size as the temperature rises as the oil starts to lower in viscosity. When the oil reaches 5° C. above the melting point of the wax, the selected wax was added and agitation and heat maintained until the wax had completely melted and all the striations had disappeared. When the mixture was completely transparent and all striations had disappeared, the heat and string bar agitator control were turned off. The string bar agitator was removed from the mixture and the mixture was allowed to cool to room temperature. When the mixture reached room temperature, it was ready to be used as a suspending medium to add probiotics or other active ingredients.

### Example 2—Preparation of Probiotic-Containing Suspension for Samples 1-28

Probiotic powders (abbreviated in all tables as “LGG”) specifically used for these examples are Lactobacillus rhamnosus GG which are available from Chr. Hansen A/S as Probio-T_{ec}® LGG® Conc. This powder was added to the oil/wax suspension media mixtures SM1-SM 12 in ratios found in Table 3 through Table 6. An IKA Eurostar® mixer was lowered into the oil/wax suspension media base and the agitation rate was slowly increased to 500 to 600 rpm. The mixing operation was performed at ambient temperature. The probiotic powders were added in their inactive form (ie. as provided in the freeze-dried or air-dried form). At all time the agitation rate was kept well below 1000 rpm so as to avoid allowing the temperature of the particle suspension to raise above 30° C.

TABLE 1
Suspension Media (SM) formulae with rice bran oil

| | SM 1 | SM 2 | SM 3 | SM 4 | SM 5 |
|---|---|---|---|---|---|
| Item | (wt %) | (wt %) | (wt %) | (wt %) | (wt %) |
| Rice bran oil | 98.0 | 97.0 | 96.0 | 98.0 | 97.0 |
| Beeswax | — | — | — | 2.0 | 3.0 |
| Carnauba wax | 2.0 | 3.0 | 4.0 | — | — |

TABLE 2
Suspension Media (SM) formulae with sunflower oil and avocado oil

| | SM 6 | SM 7 | SM 8 | SM 9 | SM 10 | SM 11 | SM 12 |
|---|---|---|---|---|---|---|---|
| Item | (wt %) | (wt %) | (wt %) | (wt %) | (wt %) | (wt %) | (wt %) |
| Sunflower oil | 98.0 | 97.0 | 98.0 | 97.0 | 95.0 | — | — |
| Avocado oil | — | — | — | — | — | 98.0 | 97.0 |
| Beeswax | — | — | 2.0 | 3.0 | 5.0 | 2.0 | 3.0 |
| Carnauba wax | 2.0 | 3.0 | — | — | — | — | — |

TABLE 3
Probiotic suspension formulae for Rice bran oil containing
oil containing suspensions

| Sample # | 1 | 2 | 3 | 4 | 5 | 6 | 7 | 8 |
|---|---|---|---|---|---|---|---|---|
| Item | (wt %) | (wt %) | (wt %) | (wt %) | (wt %) | (wt %) | (wt %) | (wt %) |
| SM 1 | 98.92 | — | — | — | — | — | — | — |
| SM 2 | — | 99.64 | 97.87 | 97.09 | 96.15 | 94.34 | 92.59 | — |
| SM 3 | — | — | — | — | — | — | — | 97.09 |
| SM 4 | — | — | — | — | — | — | — | — |
| SM 5 | — | — | — | — | — | — | — | — |
| LGG | 1.08 | 0.36 | 2.13 | 2.91 | 3.85 | 5.66 | 7.41 | 2.91 |

TABLE 4
Probiotic suspension formulae for Rice bran oil containing suspensions

| Sam- | | | | | | | |
|---|---|---|---|---|---|---|---|
| ple # | 9 | 10 | 11 | 12 | 13 | 14 | 15 |
| Item | (wt %) | (wt %) | (wt %) | (wt %) | (wt %) | (wt %) | (wt %) |
| SM 1 | — | — | — | — | — | — | — |
| SM 2 | — | — | — | — | — | — | — |
| SM 3 | 96.15 | 94.34 | 92.59 | — | — | — | — |
| SM 4 | — | — | — | 98.92 | — | — | — |
| SM 5 | — | — | — | — | 99.64 | 98.92 | 97.87 |
| LGG | 3.85 | 5.66 | 7.41 | 1.08 | 0.36 | 1.08 | 2.13 |

TABLE 5
Probiotic suspension formulae for sunflower oil and
avocado oil containing suspensions

| Sample # | 16 | 17 | 18 | 19 | 20 | 21 |
|---|---|---|---|---|---|---|
| Item | (wt %) | (wt %) | (wt %) | (wt %) | (wt %) | (wt %) |
| SM 6 | 98.92 | — | — | — | — | — |
| SM 7 | — | 99.64 | 97.87 | — | — | — |
| SM 8 | — | — | — | 98.92 | 99.64 | 97.87 |
| SM 9 | — | — | — | — | — | — |
| SM 10 | — | — | — | — | — | — |
| SM 11 | — | — | — | — | — | — |
| SM 12 | — | — | — | — | — | — |
| LGG | 1.08 | 0.36 | 2.13 | 1.08 | 0.36 | 2.13 |

TABLE 6
Probiotic suspension formulae for sunflower oil and avocado oil containing suspensions

| Sample # | 22 | 23 | 24 | 25 | 26 | 27 | 28 |
|---|---|---|---|---|---|---|---|
| Item | (wt %) | (wt %) | (wt %) | (wt %) | (wt %) | (wt %) | (wt %) |
| SM 6 | — | — | — | — | — | — | — |
| SM 7 | — | — | — | — | — | — | — |
| SM 8 | — | — | — | — | — | — | — |
| SM 9 | 97.87 | 98.92 | 99.64 | — | — | — | — |
| SM 10 | — | — | — | 98.39 | — | — | — |
| SM 11 | | | | | 98.92 | — | — |
| SM 12 | | | | | — | 99.64 | 97.87 |
| LGG | 2.13 | 1.08 | 0.36 | 1.61 | 1.08 | 0.36 | 2.13 |

### Example 3—Testing of Probiotic-Containing Suspension Samples 1-28 and Commercial Comparisons

Test results are shown in Tables 7 and 8. Testing for Table 7 focuses on the viscosities over storage time and water activities of all the probiotic-containing suspensions and comparative commercial samples. Testing for Table 8 focuses on the visual settling score of probiotic powder over storage time and the visual separation of oil and wax of all the probiotic-containing suspensions and comparative commercial samples.

TABLE 7
Testing results of probiotic suspensions for viscosity and water activity

| | 2 Week | 5 week | Water |
|---|---|---|---|
| | Viscosity | viscosity | activity |
| Sample # | @ 25° C. | @ 25° C. | (a_{w}) |
| 1 | 260 | 409 | 0.249 |
| 2 | 370 | 560 | 0.262 |
| 3 | 350 | 589 | 0.244 |
| 4 | 310 | 572 | 0.259 |
| 5 | 344 | 538 | 0.272 |
| 6 | 368 | 489 | 0.264 |
| 7 | 340 | 518 | 0.250 |
| 8 | 422 | 704 | 0.273 |
| 9 | 416 | 722 | 0.264 |
| 10 | 378 | 694 | 0.264 |
| 11 | 430 | 736 | 0.248 |
| 12 | 210 | — | 0.264 |
| 13 | 314 | — | 0.255 |
| 14 | 275 | — | 0.026 |
| 15 | 316 | — | 0.251 |
| 16 | 178 | 207 | 0.252 |
| 17 | 199 | 243 | 0.268 |
| 18 | 210 | 231 | 0.245 |
| 19 | 221 | — | 0.263 |
| 20 | 220 | — | 0.265 |
| 21 | 192 | — | 0.259 |
| 22 | 350 | — | 0.246 |
| 23 | 300 | — | 0.267 |
| 24 | 292 | — | 0.255 |
| 25 | 392 | 580 | 0.278 |
| 26 | 175 | — | 0.266 |
| 27 | 323 | — | 0.295 |
| 28 | 222 | — | 0.266 |
| Gerber's Soothe ® | 80¹ | 80¹ | 0.269 |
| probiotic drops | | | |
| Mommy's Bliss ® | 52² | 52² | 0.289 |
| probiotic drops | | | |

¹The main ingredient is sunflower oil which had a viscosity of 80 cps at 25° C. when measured using Brookfield DV-II + Pro viscometer using RV spindle #3, 100 rpm.
²The main ingredient is corn oil which had a viscosity of 52 cps at 25° C. when measured using Brookfield DV-II + Pro viscometer using RV spindle #3, 100 rpm.

TABLE 8
Testing results of probiotic suspensions for visual settling of
probiotic powder and visual separation of oil and wax

| | Visual Settling | Visual Settling | Visual |
|---|---|---|---|
| | Score of | Score | Separation |
| | probiotic after | of probiotic | of oil and |
| | 2 weeks | after 5 weeks | wax after |
| Sample # | (0-5 scale) | (0-5 scale) | 2 weeks (Yes/no) |
| 1 | 1 | 2 | No |
| 2 | 0 | 0 | No |
| 3 | 1 | 1 | No |
| 4 | 1 | 1 | No |
| 5 | 1 | 2 | No |
| 6 | 2 | 3 | No |
| 7 | 2 | 3 | No |
| 8 | 1 | 1 | No |
| 9 | 1 | 1 | No |
| 10 | 2 | 2 | No |
| 11 | 2 | 2 | No |
| 12 | 1 | — | Yes |
| 13 | 1 | — | Yes |
| 14 | 2 | — | Yes |
| 15 | 2 | — | Yes |
| 16 | 2 | 2 | Yes |
| 17 | 1 | 1 | Yes |
| 18 | 2 | 3 | Yes |
| 19 | 1 | — | No |
| 20 | 1 | — | No |
| 21 | 2 | — | Yes |
| 22 | 1 | — | No |
| 23 | 1 | — | No |
| 24 | 0 | — | Yes |
| 25 | 1 | 1 | No |
| 26 | 2 | — | Yes |
| 27 | 1 | — | No |
| 28 | 3 | — | Yes |
| Gerber's Soothe ® | 5 | 5 | — |
| probiotic drops | | | |
| Mommy's Bliss ® | 5 | 5 | — |
| probiotic drops | | | |

It can be readily seen that the probiotic powders in the commercial products Gerber's Soothe® probiotic drops and Mommy's Bliss® probiotics have already completely settled after two seeks according to the visual settling data. All samples made according to the present invention by using the combination of the edible oil and edible wax, on the other hand, are significantly improved in visual settling behavior. Furthermore, it is readily shown that water activity a_{w} has not been sacrificed for the purposes of obtaining the improved visual settling performance. In some instances, the samples show some separation between the oil and wax themselves, but even in these instances, the visual settling of the probiotic powder is still much improved.

What is claimed is:

1. A stable probiotic-containing suspension comprising:
i) at least one edible oil;
ii) at least one edible wax; and
iii) a probiotic powder;
wherein the stable probiotic-containing suspension has a visual settling score of 3 or less when stored for more than one week at 0° C. to 40° C.; and wherein the water activity of the stable probiotic-containing suspension is between 0.001 to 0.40 a_{w} when run in the water activity test.

2. The stable probiotic-containing suspension according to claim 1, wherein i)-iii) are all fit for human consumption in their composition and purity levels;

3. The stable probiotic-containing suspension according to claim 2, wherein the stable probiotic-containing suspension has a visual settling score of 3 or less when stored for more than five weeks at 0° C. to 40° C.

4. The stable probiotic-containing suspension according to any one of claims 1-3, wherein the edible oil is selected from the group of rice bran oil, sunflower oil, corn oil, canola oil, avocado oil, olive oil, safflower oil, soybean oil, peanut oil, sesame oil, cottonseed oil, coconut oil, grape seed oil, palm oil, flaxseed oil, rapeseed oil, or combinations thereof.

5. The stable probiotic-containing suspension according to any one of claims 1-4, wherein the edible wax is selected from the group of carnauba wax, beeswax, candelilla wax, ceresine wax, microcrystalline wax, ozokerite wax, paraffin wax, cetyl palmitate, or combinations thereof.

6. The stable probiotic-containing suspension according to any one of claims 1-5, wherein the probiotic powder is selected from the group of Lactobacillus, Bifidobacterium, Bacillus, Streptococcus, Saccharomyces, Enterococcus, Staphylococcus, or combinations thereof.

7. The stable probiotic-containing suspension according to any one of claims 1-6, wherein the a_{w} is between 0.001 to 0.35 a_{w}.

8. The stable probiotic-containing suspension according to any one of claims 1-6, wherein the a_{w} is between 0.001 to 0.30 a_{w}.

9. The stable probiotic-containing suspension according to any one of claims 1-8, wherein viscosity of the stable probiotic-containing suspension is between 100 to 100000 centipoises.

10. The stable probiotic suspension according to any one of claims 1-8, wherein viscosity of the stable probiotic-containing suspension is between 100 to 6000 centipoises.

11. The stable probiotic-containing suspension according to any one of claims 1-8, wherein viscosity of the stable probiotic-containing suspension is between 100 to 3000 centipoises.

12. A stable probiotic-containing suspension comprising:
i) at least one edible oil;
ii) at least one edible shear thickening additive; and
iii) a probiotic powder;
wherein the stable probiotic-containing suspension has a visual settling score of 3 or less when stored for more than one week at 0° C. to 40° C.; and wherein the water activity of the stable probiotic-containing suspension is between 0.001 to 0.40 a_{w}.

13. The stable probiotic-containing suspension according to claim 12, wherein i)-iii) are all fit for human consumption in their composition and purity levels;

14. The stable probiotic-containing suspension according to claim 13, wherein the stable probiotic-containing suspension has a visual settling score of 3 or less when stored for more than five weeks at 0° C. to 40° C.

15. The stable probiotic-containing suspension according to any one of claims 12-14, wherein the edible oil is selected from the group of rice bran oil, sunflower oil, corn oil, canola oil, avocado oil, olive oil, safflower oil, soybean oil, peanut oil, sesame oil, cottonseed oil, coconut oil, grape seed oil, palm oil, flaxseed oil, rapeseed oil, or combinations thereof.

16. The stable probiotic-containing suspension according to any one of claims 12-15, wherein the edible shear thickening additive is selected from the group of gums, polysaccharides, collagens, starches, or combinations thereof.

17. The stable probiotic-containing suspension according to any one of claims 12-16 wherein the probiotic powder is selected from the group of Lactobacillus, Bifidobacterium, Bacillus, Streptococcus, Saccharomyces, Enterococcus, Staphylococcus, or combinations thereof.

18. The stable probiotic-containing suspension according to any one of claims 12-17 wherein the a_{w} is between 0.01 to 0.35 a_{w}.

19. The stable probiotic-containing suspension according to any one of claims 2-17 wherein the a_{w} is between 0.01 to 0.30 a_{w}

20. The stable probiotic-containing suspension according to any one of claims 12-19 wherein viscosity of the stable probiotic suspension is between 100 to 100000 centipoises.

21. The stable probiotic-containing suspension according to any one of claims 12-19 wherein viscosity of the stable probiotic suspension is between 100 to 6000 centipoises.

22. The stable probiotic-containing suspension according to any one of claims 12-19 wherein viscosity of the stable probiotic suspension is between 100 to 3000 centipoises.

23. A method of making the stable probiotic-containing suspension according to any one of claims 1-11, comprising the steps of:
a. Adding an edible oil to a mixing vessel;
b. Heating the vessel to above the melting point of the edible wax;
c. Adding the edible wax to the mixing vessel;
d. Agitating with an appropriate agitator until the oil and wax mixture is homogenous and shows no visible striations;
e. Cooling the oil and wax mixture to below 30° C.;
f. Adding a probiotic powder; and
g. Agitating the mixture until the probiotic powder is well suspended.

24. The method of claim 23, wherein in step b the vessel is heated to at least 5° C. above the melting point of an edible wax.

25. A method of making the stable probiotic-containing suspension according to any one of claims 1-11, comprising the steps of:
a. Adding a 5-99% of the total amount of edible oil to be used to a mixing vessel;
b. Heating the vessel to above the melting point of an edible wax;
c. Adding the edible wax to the mixing vessel;
d. Agitating with an appropriate agitator until the oil and wax mixture is homogenous and shows no visible striations;
e. Add the remaining 95-1% edible oil to be used to the mixing vessel to decrease the temperature of the oil/wax mixture, continue to mix until mixture becomes homogeneous.
f. Cooling the oil and wax mixture to below 30° C.;
g. Adding a probiotic powder; and
h. Agitating the mixture until the probiotic powder is well suspended.

26. The method of claim 25, wherein in step b the vessel is heated to at least 5° C. above the melting point of an edible wax.
